(19) United States
(12) Patent Application Publication
(10) Pub. No.: US 2025/0353632 A1
(43) Pub. Date: Nov. 20, 2025

(54) REMOTE TRACKING AND APPROVAL OF MEDICATION DELIVERY

(71) Applicant: OPIO CONNECT, INC., Kalispell, MT (US)

(72) Inventors: Amber Norbeck, Kalispell, MT (US); Michael Pokorny, Kalispell, MT (US); Kelly Chu, Kalispell, MT (US); Thomas Hoffmann, Kalispell, MT (US)

(21) Appl. No.: 19/211,700

(22) Filed: May 19, 2025

Related U.S. Application Data
(60) Provisional application No. 63/648,895, filed on May 17, 2024.

(51) Int. Cl. B65B 57/10; A61J 1/16; B65B 5/06; B65B 5/10; B65B 61/02; G16H 20/13; H04N 7/18

(52) U.S. Cl. CPC B65B 57/10; A61J 1/16; B65B 5/068; B65B 5/103; B65B 61/025; A61J 2200/70; A61J 2205/30; G16H 20/13; H04N 7/181; H04N 7/183

(57) ABSTRACT

A system for dispensing and verifying a dosage in a bottle. The system includes a stage for holding the bottle. The system further includes a pump-printer module, configured to receive a set of dosage specifications. The module includes a dosage dispenser configured to fill the bottle with the dosage according to the set of dosage specifications, and a printer device configured to print and apply a label onto the bottle listing out the set of dosage specifications. The dosage dispenser and the printer device are configured to operate simultaneously. The system further includes one or more imaging components disposed optically in-line with the stage, configured to capture a plurality of images of the bottle and the label.

### CROSS-REFERENCE TO RELATED APPLICATIONS

This application is a non-provisional and claims benefit of U.S. Provisional Application No. 63/648,895 filed May 17, 2024, the specification of which is incorporated herein in its entirety by reference.

### FIELD OF THE INVENTION

The present invention is directed to remote dispensing and verification of controlled substances and pharmaceutical drugs.

### BACKGROUND OF THE INVENTION

Pharmaceutical drugs and controlled substances used to treat various ailments and addictions require comprehensive tracking and dose verification by authorized medical officials before being dispensed to patients. This is to prevent delivering an incorrect dosage type or amount to the patient, as well as preventing any tampering with the bottle for illicit purposes between when the dosage is dispensed and delivered. This verification process is time-consuming and is a major bottleneck for the delivery of treatment doses to patients. Thus, there exists a present need for a high-throughput and efficient method for dispensing and verifying doses of controlled substances and pharmaceutical drugs.

### BRIEF SUMMARY OF THE INVENTION

It is an objective of the present invention to provide systems that allow for remote dispensing and verification of controlled substances and pharmaceutical drugs, as specified in the independent claims. Embodiments of the invention are given in the dependent claims. Embodiments of the present invention can be freely combined with each other if they are not mutually exclusive.

The present invention features a system for dispensing and verifying a dosage in a bottle. In some embodiments, the system may comprise a stage. The bottle may be disposed on the stage. The system may further comprise a pump-printer module operatively coupled to the stage, configured to receive a set of dosage specifications. In some embodiments, the module may comprise a dosage dispenser configured to fill the bottle with the dosage according to the set of dosage specifications, and a printer device configured to print and apply a label onto the bottle, the label comprising the set of dosage specifications. The dosage dispenser and the printer device may be configured to operate simultaneously. The system may further comprise one or more imaging components disposed optically in-line with the stage, configured to capture a plurality of images of the bottle and the label.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a view of a tabletop implementation of the remote drug dispensing and verification system of the present invention.

FIG. 2 shows a view of an automated implementation of the remote drug dispensing and verification system of the present invention.

FIG. 3 shows a close-up view of an imaging component of the present invention.

### DETAILED DESCRIPTION OF THE INVENTION

Following is a list of elements corresponding to a particular element referred to herein:
100 system
110 stage
120 pump-printer module
122 dosage dispenser
124 printer device
130 imaging components
140 computing system
150 bottle-detecting sensor
160 automated arm

The term “stage” is defined herein as a surface upon which a bottle can be placed.

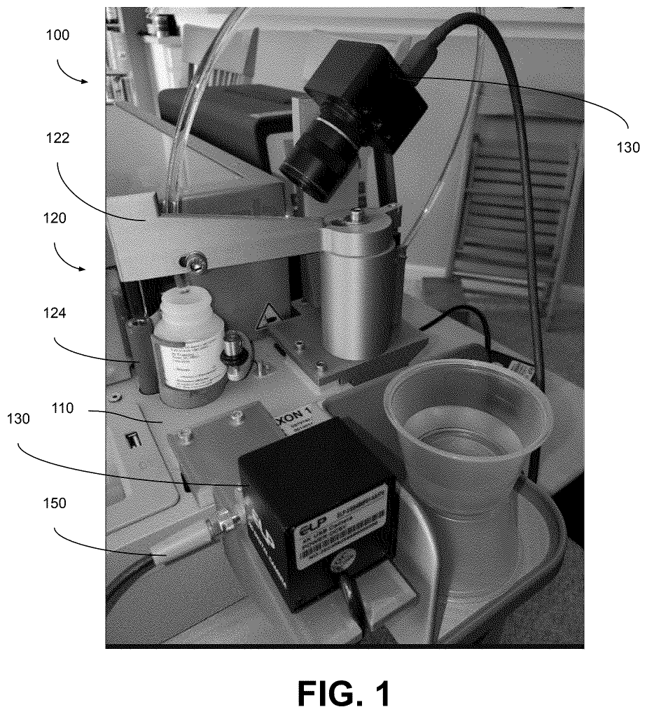

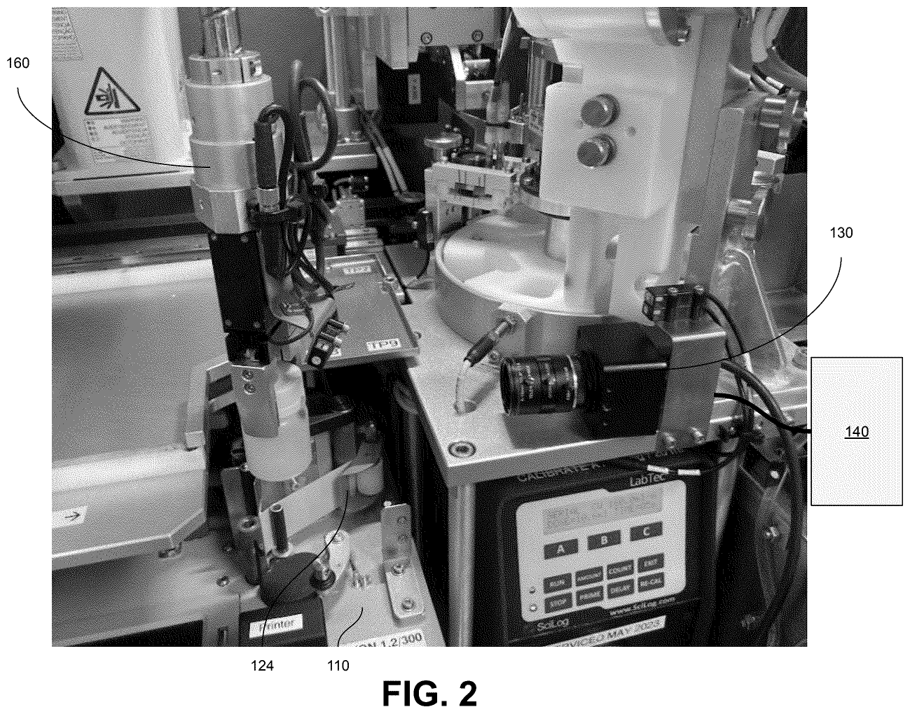

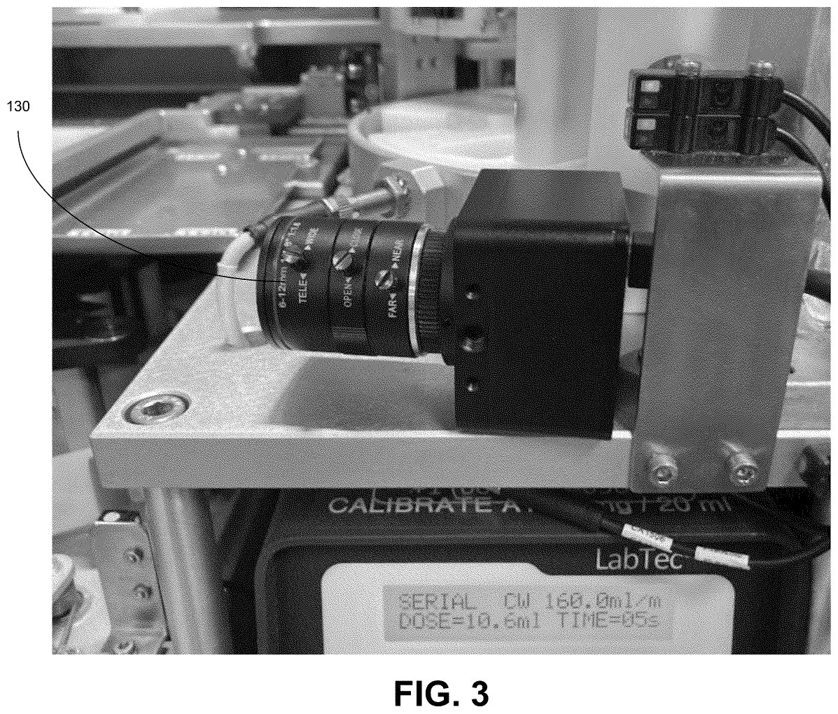

Referring now to FIGS. 1-3, the present invention features a system (100) for dispensing and verifying a dosage in a bottle. In some embodiments, the system (100) may comprise a stage (110). The bottle may be disposed on the stage (110). The system (100) may further comprise a pump-printer module (120) operatively coupled to the stage (110), configured to receive a set of dosage specifications. In some embodiments, the module (120) may comprise a dosage dispenser (122) configured to fill the bottle with the dosage according to the set of dosage specifications, and a printer device (124) configured to print and apply a label onto the bottle, the label comprising the set of dosage specifications. The dosage dispenser (122) and the printer device (124) may be configured to operate simultaneously. The system (100) may further comprise one or more imaging components (130) disposed optically in-line with the stage (110), configured to capture a plurality of images of the bottle and the label.

In some embodiments, the system may further comprise a bottle-detecting sensor (150) operatively coupled to the stage (110), configured to detect whether or not the bottle is present on the stage (110). In some embodiments, the pump-printer module (120) may be configured to actuate when the bottle-detecting sensor (150) detects the bottle on the stage (110). In some embodiments, the automated arm (160) may be configured to place a new bottle on the stage (110) when the bottle-detecting sensor (150) detects that there isn't a bottle on the stage (110). In some embodiments, the printer device (124) may comprise a printing component configured to print the set of dosage specifications onto the label, and a rolling mechanism configured to roll the label around a circumference of the bottle such that the label adheres to the bottle.

In some embodiments, the one or more imaging components (130) may comprise a single camera operatively coupled to a rotation mechanism configured to rotate the single camera to a plurality of points optically in-line with the stage (110) such that the plurality of images comprise images of the bottle and the label at a plurality of angles. In some embodiments, the one or more imaging components (130) may comprise a plurality of cameras disposed at a plurality of points optically in-line with the stage (110) such that the plurality of images comprise images of the bottle and the label at a plurality of angles. In some embodiments, the one or more imaging components (130) may comprise one or more standard cameras, one or more video cameras, or a combination thereof.

In some embodiments, the set of dosage specifications may comprise a dose amount, a type of drug, or a combination thereof. In some embodiments, the system (100) may further comprise an automated arm (160) operatively coupled to the stage (110), communicatively coupled to the one or more imaging components (130), configured to place the bottle on the stage (110), remove the bottle from the stage (110) after the plurality of images are taken by the one or more imaging components (130), or a combination thereof. In some embodiments, the automated arm (160) may be further configured to place the bottle in a locking tray after removing the bottle from the stage (110). The locking tray may be configured to store the bottle after receiving the bottle from the automated arm (160).

The present invention features a system (100) for dispensing and verifying a dosage in a bottle. In some embodiments, the system (100) may comprise a stage (110). The bottle may be disposed on the stage (110). The system (100) may further comprise a pump-printer module (120) operatively coupled to the stage (110), configured to receive a set of dosage specifications. In some embodiments, the module (120) may comprise a dosage dispenser (122) configured to fill the bottle with the dosage according to the set of dosage specifications. The module (120) may further comprise a printer device (124) configured to print and apply a label onto the bottle, the label comprising the set of dosage specifications. The dosage dispenser (122) and the printer device (124) may be configured to operate simultaneously. The system (100) may further comprise one or more imaging components (130) disposed optically in-line with the stage (110), configured to capture a plurality of images of the bottle and the label.

The system (100) may further comprise a computing system (140) communicatively coupled to the pump-printer module (120) and the one or more imaging components (130), comprising a processor configured to execute computer-readable instructions, and a memory component operatively coupled to the processor. The memory component may comprise computer-readable instructions for receiving the dosage specifications from an external source. The computer-readable instructions may further comprise transmitting the dosage specifications to the pump-printer module (120). The computer-readable instructions may further comprise receiving the plurality of images from the one or more imaging components (130). The computer-readable instructions may further comprise transmitting the plurality of images to an authorized medical official. The computer-readable instructions may further comprise receiving verification of the dosage from the authorized medical official based on the plurality of images.

In some embodiments, the computer-readable instructions may further comprise receiving a dosage request from the external source, wherein the dosage request comprises the set of dosage specifications, and associating the plurality of images with the dosage request. In some embodiments, the computer-readable instructions may further comprise associating a timestamp, a description, or a combination thereof with each image of the plurality of images. In some embodiments, the dosage request may be associated with a medical record.

In some embodiments, the authorized medical official may comprise a nurse, a pharmacist, or a doctor. In some embodiments, the system of the present invention may be a stand-alone tabletop device. In some embodiments, the system of the present invention may be integrated into an automated device for dosage pumping. In some embodiments, the pump-printer module may be actuated by an actuation component (e.g. a push button).

In some embodiments, verifying the plurality of images may comprise receiving the images from the system at a remote computing device communicatively coupled to the computing system. The official may view the images to analyze the bottle to verify the amount of medicine in the bottle and to analyze the label to verify that the information on the label matches up with the dosage request and/or the medical record. Upon verifying this information, the official may transmit the verification back to the computing system.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system (100) for dispensing and verifying a dosage in a bottle, the system (100) comprising:
a. a stage (110), wherein the bottle is disposed on the stage (110);
b. a pump-printer module (120) operatively coupled to the stage (110), configured to receive a set of dosage specifications, the module (120) comprising:
i. a dosage dispenser (122) configured to fill the bottle with the dosage according to the set of dosage specifications; and
ii. a printer device (124) configured to print and apply a label onto the bottle, the label comprising the set of dosage specifications;
wherein the dosage dispenser (122) and the printer device (124) are configured to operate simultaneously; and
c. one or more imaging components (130) disposed optically in-line with the stage (110), configured to capture a plurality of images of the bottle and the label.

2. The system (100) of claim 1 further comprising a bottle-detecting sensor (150) operatively coupled to the stage (110), configured to detect whether or not the bottle is present on the stage (110).

3. The system (100) of claim 2, wherein the pump-printer module (120) is configured to actuate when the bottle-detecting sensor (150) detects the bottle on the stage (110).

4. The system (100) of claim 1, wherein the printer device (124) comprises a printing component configured to print the set of dosage specifications onto the label, and a rolling mechanism configured to roll the label around a circumference of the bottle such that the label adheres to the bottle.

5. The system (100) of claim 1, wherein the one or more imaging components (130) comprise a single camera operatively coupled to a rotation mechanism configured to rotate the single camera to a plurality of points optically in-line with the stage (110) such that the plurality of images comprise images of the bottle and the label at a plurality of angles.

6. The system (100) of claim 1, wherein the one or more imaging components (130) comprise a plurality of cameras disposed at a plurality of points optically in-line with the stage (110) such that the plurality of images comprise images of the bottle and the label at a plurality of angles.

7. The system (100) of claim 1, wherein the one or more imaging components (130) comprise one or more standard cameras, one or more video cameras, or a combination thereof.

8. The system (100) of claim 1, wherein the set of dosage specifications comprise a dose amount, a type of drug, or a combination thereof.

9. The system (100) of claim 1 further comprising an automated arm (160) operatively coupled to the stage (110), communicatively coupled to the one or more imaging components (130), configured to place the bottle on the stage (110), remove the bottle from the stage (110) after the plurality of images are taken by the one or more imaging components (130), or a combination thereof.

10. The system (100) of claim 9, wherein the automated arm (160) is further configured to place the bottle in a locking tray after removing the bottle from the stage (110), wherein the locking tray is configured to store the bottle after receiving the bottle from the automated arm (160).

11. A system (100) for dispensing and verifying a dosage in a bottle, the system (100) comprising:
a. a stage (110), wherein the bottle is disposed on the stage (110);
b. a pump-printer module (120) operatively coupled to the stage (110), configured to receive a set of dosage specifications, the module (120) comprising:
i. a dosage dispenser (122) configured to fill the bottle with the dosage according to the set of dosage specifications; and
ii. a printer device (124) configured to print and apply a label onto the bottle, the label comprising the set of dosage specifications;
wherein the dosage dispenser (122) and the printer device (124) are configured to operate simultaneously;
c. one or more imaging components (130) disposed optically in-line with the stage (110), configured to capture a plurality of images of the bottle and the label; and
d. a computing system (140) communicatively coupled to the pump-printer module (120) and the one or more imaging components (130), comprising a processor configured to execute computer-readable instructions, and a memory component operatively coupled to the processor, comprising computer-readable instructions for:
i. receiving the dosage specifications from an external source;
ii. transmitting the dosage specifications to the pump-printer module (120);
iii. receiving the plurality of images from the one or more imaging components (130);
iv. transmitting the plurality of images to an authorized medical official; and
v. receiving verification of the dosage from the authorized medical official based on the plurality of images.

12. The system (100) of claim 11, wherein the printer device (124) comprises a printing component configured to print the set of dosage specifications onto the label, and a rolling mechanism configured to roll the label around a circumference of the bottle such that the label adheres to the bottle.

13. The system (100) of claim 11, wherein the one or more imaging components (130) comprise a single camera operatively coupled to a rotation mechanism configured to rotate the single camera to a plurality of points optically in-line with the stage (110) such that the plurality of images comprise images of the bottle and the label at a plurality of angles.

14. The system (100) of claim 11, wherein the one or more imaging components (130) comprise a plurality of cameras disposed at a plurality of points optically in-line with the stage (110) such that the plurality of images comprise images of the bottle and the label at a plurality of angles.

15. The system (100) of claim 11 further comprising an automated arm (160) operatively coupled to the stage (110), communicatively coupled to the one or more imaging components (130), configured to place the bottle on the stage (110), remove the bottle from the stage (110) after the plurality of images are taken by the one or more imaging components (130), or a combination thereof.

16. The system (100) of claim 11, wherein the computer-readable instructions further comprise:
a. receiving a dosage request from the external source, wherein the dosage request comprises the set of dosage specifications; and
b. associating the plurality of images with the dosage request.

17. The system (100) of claim 16, wherein the computer-readable instructions further comprise associating a timestamp, a description, or a combination thereof with each image of the plurality of images.

18. The system (100) of claim 16, wherein the dosage request is associated with a medical record.

19. A system (100) for dispensing and verifying a dosage in a bottle, the system (100) comprising:
a. a stage (110), wherein the bottle is disposed on the stage (110);
b. a bottle-detecting sensor (150) operatively coupled to the stage (110), configured to detect whether or not the bottle is present on the stage (110);
c. a pump-printer module (120) operatively coupled to the stage (110) and the bottle-detecting sensor (150), configured to receive a set of dosage specifications and actuate when the bottle-detecting sensor (150) detects the bottle on the stage (110), the module (120) comprising:
i. a dosage dispenser (122) configured to fill the bottle with the dosage according to the set of dosage specifications; and
ii. a printer device (124) configured to print and apply a label onto the bottle, the label comprising the set of dosage specifications, the printer device (124) comprising:
A. a printing component configured to print the set of dosage specifications onto the label; and
B. a rolling mechanism configured to roll the label around a circumference of the bottle such that the label adheres to the bottle;
wherein the dosage dispenser (122) and the printer device (124) are configured to operate simultaneously;
d. one or more imaging components (130) disposed optically in-line with the stage (110), configured to capture a plurality of images of the bottle and the label; and
e. a computing system (140) communicatively coupled to the pump-printer module (120) and the one or more imaging components (130), comprising a processor configured to execute computer-readable instructions, and a memory component operatively coupled to the processor, comprising computer-readable instructions for:
i. receiving a dosage request associated with a medical record from an external source, wherein the dosage request comprises the set of dosage specifications;
ii. transmitting the dosage specifications to the pump-printer module (120);
iii. receiving the plurality of images from the one or more imaging components (130);
iv. transmitting the plurality of images to an authorized medical official;
v. receiving verification of the dosage from the authorized medical official based on the plurality of images; and
vi. associating the plurality of images with the dosage request.
